(19) United States
(12) Patent Application Publication
(10) Pub. No.: US 2024/0319101 A1
(43) Pub. Date: Sep. 26, 2024

(54) MULTIFUNCTIONAL HOLLOW SILICA NANOFIBER EXTRACELLULAR MATRIX

(71) Applicant: Trustees of Dartmouth College, Hanover, NH (US)

(72) Inventors: John X.J. Zhang, Hanover, NH (US); Junhu Zhou, Hanover, NH (US); Yundong Ren, Hanover, NH (US)

(73) Assignee: Trustees of Dartmouth College, Hanover, NH (US)

(21) Appl. No.: 18/578,674

(22) PCT Filed: Jun. 11, 2022
(86) PCT No.: PCT/US2022/033156

§ 371 Date: Jan. 11, 2024

Related U.S. Application Data
(60) Provisional application No. 63/220,594, filed on Jul. 12, 2021.

(51) Int. Cl. G01N 21/77; G01N 21/63

(52) U.S. Cl. CPC G01N 21/7703; G01N 21/63

(57) ABSTRACT

Embodiments of the present disclosure pertain to a composition that includes: silica nanofibers; and one or more detection particles associated with the silica nanofibers. Additional embodiments of the present disclosure pertain to methods of sensing one or more analytes and/or one or more environmental conditions from a sample by associating the sample with a composition of the present disclosure; detecting a change in a property of the detection particles; and correlating the change in the property of the detection particles to a presence or absence of one or more analytes, one or more environmental conditions, or combinations thereof. Further embodiments of the present disclosure pertain to methods of making the compositions of the present disclosure by growing silica nanofibers from at least one precursor material; and associating one or more detection particles with the silica nanofibers.

### CROSS-REFERENCE TO RELATED APPLICATIONS

This application claims priority to U.S. Provisional Pat. App. No. 63/220,594, filed on Jul. 12, 2021. The entirety of the aforementioned application is incorporated herein by reference.

### BACKGROUND

Current sensors suffer from numerous limitations, such as in the multiplexed sensing of different analytes and environmental conditions. Various embodiments of the present disclosure seek to address these limitations.

### SUMMARY

In some embodiments, the present disclosure pertains to a composition that includes: silica nanofibers; and one or more detection particles associated with the silica nanofibers. In some embodiments, at least one of the one or more detection particles are operational to exhibit a change in a property upon interaction with one or more analytes, upon detection of one or more environmental conditions, or combinations thereof. In some embodiments, the change in property includes, without limitation, a change in optical intensity, a change in emission wavelength peak, a change in emission wavelength phase, fluorescence resonance energy transfer (FRET), a shift in localized surface plasmonic resonances (LSPR), and combinations thereof.

In some embodiments, the compositions of the present disclosure are a component of a sensor. In some embodiments, the sensor includes: a surface associated with the silica nanofibers; a light source positioned near the silica nanofibers and operational to transmit light to the detection particles associated with the silica nanofibers; and a measuring device operational to measure light emitted from the detection particles associated with the silica nanofibers. In some embodiments, the sensor also includes an optical fiber that is connected to the measuring device and positioned near the silica nanofibers.

Further embodiments of the present disclosure pertain to methods of sensing one or more analytes and/or one or more environmental conditions from a sample. In some embodiments, the methods of the present disclosure include the following steps: associating the sample with a composition of the present disclosure, where the composition includes silica nanofibers and one or more detection particles associated with the silica nanofibers; detecting a change in a property of the detection particles; and correlating the change in the property of the detection particles to a presence or absence of one or more analytes, one or more environmental conditions, or combinations thereof.

Additional embodiments of the present disclosure pertain to methods of making the compositions of the present disclosure. In some embodiments, the methods of the present disclosure include: growing silica nanofibers from at least one precursor material; and associating one or more detection particles with the silica nanofibers.

### DESCRIPTION OF THE DRAWINGS

FIG. 1A depicts a composition according to an aspect of the present disclosure.

FIG. 1B depicted a sensor according to an aspect of the present disclosure.

FIG. 1C illustrates a method of sensing an analyte or an environmental condition from a sample according to an aspect of the present disclosure.

FIG. 1D illustrates a method of making a composition according to an aspect of the present disclosure.

FIG. 2 provides a schematic of a hollow silica nanofiber (hSNF) pH sensor and the optical characterization setup in accordance with various embodiments of the present disclosure. The left upper inset is a microscope image of the tilted optical fiber tip positioned ˜3 μm above the hSNFs.

FIGS. 3A-3E provide schematics of SNF synthesis, surface coating and deposition processes.

FIGS. 4A-4I provide characterization images of hSNFs. FIGS. 4A-4C provide scanning electron microscopy (SEM) images of hSNFs, including hSNFs before (FIG. 4B) and after (FIG. 4C) FITC coating. FIGS. 4D-4F provide transmission electron microscopy (TEM) images of hSNFs at different scales. FIGS. 4G-4H show fluorescence images of FITC and Ru(BPY)₃. FIG. 41 shows bright filed images of hSNFs on polystyrene substrates.

FIGS. 5A-5F provide energy-dispersive X-ray spectroscopy (EDS) characterization of dual fluorescence hSNFs. FIGS. 5A-5B show the EDS energy spectrum of hSNFs (FIG. 5A) and its enlarged view (FIG. 5B) showing the peaks of element S and Ru. FIG. 5C shows a field of view of EDS map. Also shown are EDS elemental mapping of Si (FIG. 5D), S (FIG. 5E) and Ru (FIG. 5F).

FIGS. 6A-6C show various emission spectra of hSNFs. FIG. 6A shows emission spectra of FITC dissolved in water and FITC coated hSNFs dissolved in water. FIG. 6B shows emission spectra of Ru(BPY)₃ dissolved in water and Ru(BPY)₃ coated hSNFs dissolved in water. FIG. 6C shows emission spectra of hSNFs coated with both FITC and Ru(BPY)₃.

FIGS. 7A-D provide characterization of the pH response of the dual fluorescence hSNFs. FIG. 7A provides normalized optical spectra of the dual fluorescence hSNFs measured in solutions of different pH. The plot legends show the correspondence between the line color and the pH value. FIG. 7B shows linear fitting of the I_{FITC}/I_{Ru(BPY)3} against different pH values. FIG. 7C shows normalized optical spectra measured in solutions with acidic, alkaline, and intermedia pH values. Different shades of red and blue and gray separate the spectra taken at different pH and at different times. FIG. 7D shows I_{FITC}/I_{Ru(BPY)3} calculated from the spectra in FIG. 7C plotted against the elapsed time.

FIGS. 8A-8D provide the characterization of the stability of the dual fluorescence hSNFs. FIG. 8A shows the plot of I_{FITC}/I_{Ru(BPY)3} at different elapsed times calculated from the spectra in FIG. 8B. FIG. 8B shows normalized optical spectra of the dual fluorescence hSNFs measured in the pH=7 buffer solution over 6 hours. The different shades of red, green, blue, and black color of the spectra represent the different times at which the spectra were taken.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Current sensors, such as those utilized in localized surface plasmon resonance (LSPR) and fluorescence, suffer from numerous disadvantages. For instance, real-time pH measurements are of great importance in chemical reactions, environmental monitoring, and physiological processes. Measuring pH values, especially for that in small volumes, is difficult but necessary for a variety of scientific applications.

In particular, it is well known that the local microenvironment pH has great influence on immunization, cell metabolism, and enzyme activity. Commercially available glass electrode pH sensors are widely used in laboratories for pH measurements. However, the glass electrode pH sensors have the disadvantages for limited accessibility of low volume detection, show slow drift, and are unsuitable for long-term pH monitoring. Furthermore, the glass-electrode-based pH probes have an inherent measurement error on the order of 0.1 pH unit, which limits their uses in precision measurements.

Optical pH sensors have proved to be an ideal alternative because of its advantages including rapid response, low sample volume, and the possibility of contactless measurement. Many optical transducers such as organic molecules, have been widely used to monitor local pH values. These organic molecules' fluorescence intensity changes when the environmental pH varies. Examples of such organic molecules include fluorophores like Fluorescein, Rhodamine, Oregon Green 488, and Coumarin. However, the accuracy of the pH measurement based on the single fluorescence emission intensity change are susceptible to both extrinsic and intrinsic factors, such as the stability of the excitation light, the concentration of the organic molecules, and the photobleaching of the organic molecules.

Therefore, efforts have been made to enhance the repeatability of the fluorescence pH sensors using ratiometric detections, which can eliminate or alleviate the environmental factors' influence on the accuracy of the measurements. The ratiometric sensors have fluorescence emissions with peaks of two different wavelengths. These emission peaks can be from different dyes or from the same dye that has two different emission wavelengths. To achieve ratiometric measurements, one of the fluorescence emission peak intensities should be sensitive to the measurand, while the other emission peak intensity should be insensitive to the measurand and be used as the reference signal.

By normalizing the measurand-sensitive peak intensity change with the measurand-insensitive peak intensity, the ratiometric measurements provide self-calibrated results with much better reliability. Specifically for pH sensing, the combination of the pH responsive dye and the reference dye can be carefully designed so that they can be excited by a single light source and the emission peak wavelengths are separated as far as possible to avoid signal distortions.

Silica is an intrinsically non-toxic, environmentally friendly material possessing great mechanical strength. Silica is widely used in sensing applications due to the solid encapsulating molecules by virtue of the rigidity of the inorganic cage. The use of silica-based material for pH probes embedding and immobilization has been demonstrated. In most studies, pH-probes are embedded in silica nanoparticles due to the unique properties, such as large specific surface area and thermal stability. However, most of these nanosensors are used for imaging or intracellular pH monitoring.

As such, a need exists for more effective systems and methods for sensing analytes and environmental conditions. Various embodiments of the present disclosure address the aforementioned need.

### Compositions

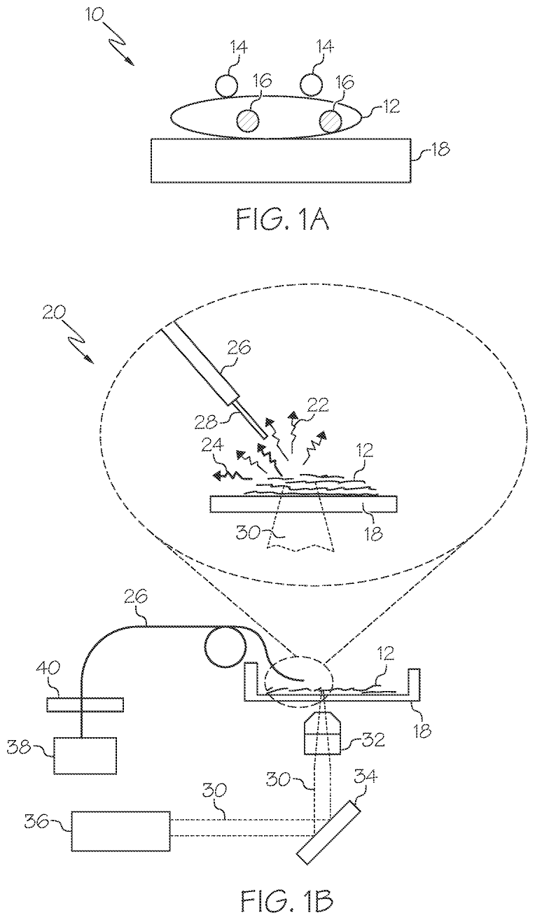

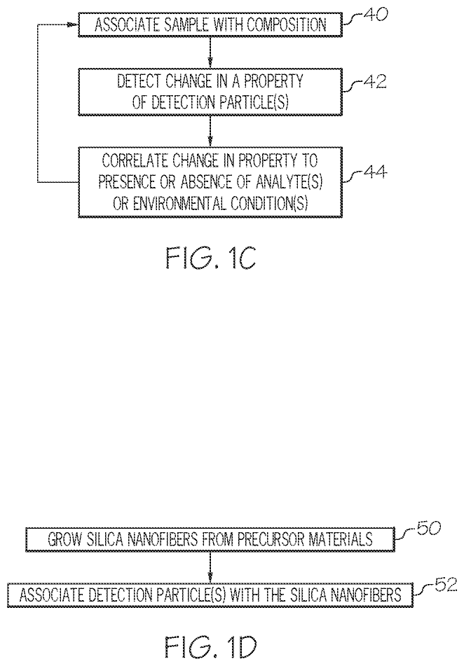

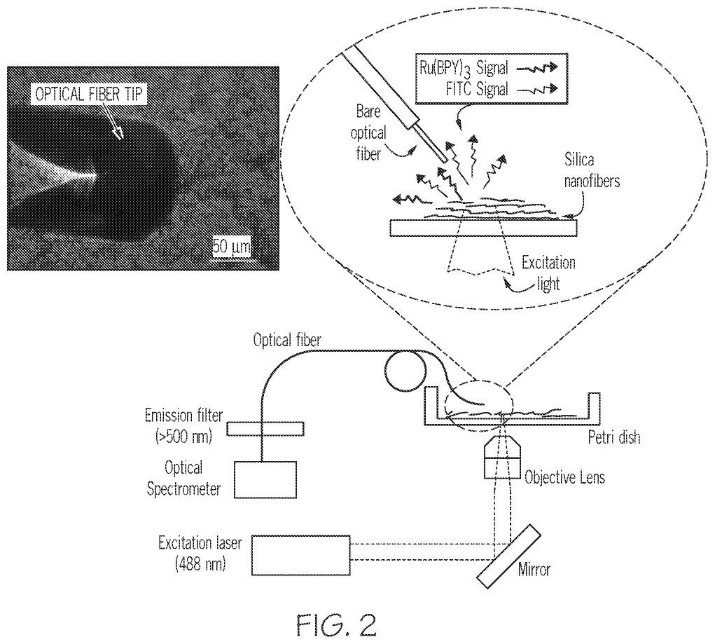

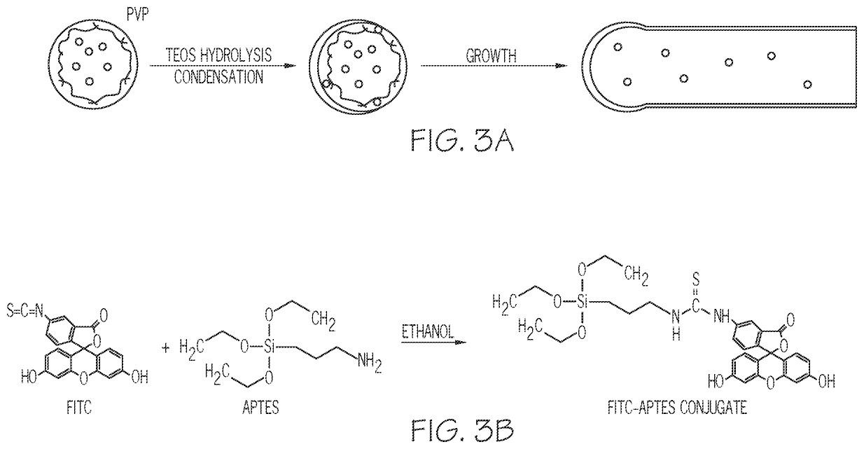

In some embodiments, the present disclosure pertains to a composition that includes silica nanofibers and one or more detection particles associated with the silica nanofibers. In some embodiments illustrated in FIG. 1A, the compositions of the present disclosure are in the form of composition 10, which includes silica nanofibers 10 and detection particles 14 and 16 associated with the silica nanofibers. In this example, silica nanofibers include a hollow cavity. Additionally, detection particles 14 are on a surface of the silica nanofibers while detection particles 16 are within the hollow cavity of the silica nanofibers. Additionally, silica nanofibers are positioned on surface 18. As set forth in more detail herein, the compositions of the present disclosure can have numerous embodiments.

### Silica Nanofibers

The compositions of the present disclosure can include various types of silica nanofibers. For instance, in some embodiments, the silica nanofibers of the present disclosure are in the form of a matrix. In some embodiments, the silica nanofibers are in the form of an extracellular matrix (ECM). In some embodiments, the ECM is a multifunctional ECM. In some embodiments, the silica nanofibers of the present disclosure are composed of silicon dioxide. In some embodiments, the silica nanofibers of the present disclosure have a nanoscale structure.

The silica nanofibers of the present disclosure can also include various sizes and geometries. For instance, in some embodiments, the silica nanofibers of the present disclosure include a length of at least about 500 nm and a diameter of at least about 50 nm. In some embodiments, the silica nanofibers of the present disclosure include a length of at least about 1 m and a diameter of at least about 100 nm. In some embodiments, the silica nanofibers of the present disclosure include a length of at least about 1 m and a diameter of at least about 400 nm. In some embodiments, the silica nanofibers of the present disclosure include a length of at least about 1 m and a diameter of about 410 nm.

In some embodiments, the silica nanofibers of the present disclosure have a length to width aspect ratio of at least about 10. In some embodiments, the silica nanofibers of the present disclosure have a length to width aspect ratio of at least about 20. In some embodiments, the silica nanofibers of the present disclosure have a length to width aspect ratio of at least about 30. In some embodiments, the silica nanofibers of the present disclosure have a length to width aspect ratio ranging from about 10 to about 30. In some embodiments, the silica nanofibers of the present disclosure include a hollow cavity.

### Detection Particles

Detection particles generally refer to particles that are operational to exhibit a change in a property upon interaction with one or more analytes, upon detection of one or more environmental conditions, or combinations thereof. In some embodiments, the change in property includes, without limitation, a change in optical intensity, a change in emission wavelength peak, a change in emission wavelength phase, fluorescence resonance energy transfer (FRET), a shift in localized surface plasmonic resonances (LSPR), and combinations thereof.

The detection particles of the present disclosure may be associated with silica nanofibers in various manners. For instance, in some embodiments, the detection particles are on an outer surface of the silica nanofibers (e.g., detection particles 14 illustrated in FIG. 1A). In some embodiments, the detection particles are within a hollow cavity of the silica nanofibers (e.g., detection particles 16 illustrated in FIG. 1A). In some embodiments, the detection particles are on an outer surface of the silica nanofibers and within a hollow cavity of the silica nanofibers.

The silica nanofibers of the present disclosure may be associated with various types of detection particles. For instance, in some embodiments, the detection particles include, without limitation, chromophores, fluorophores, plasmonic nanoparticles, Ru(2,2′-bipyridine)₃ (Ru(bpy)₃), fluorescein isothiocyanate (FITC), quantum dots, and combinations thereof. In some embodiments, the detection particles include FITC. In some embodiments, the detection particles include Ru(bpy)₃. In some embodiments, the detection particles include FITC and Ru(bpy)₃.

In some embodiments, the detection particles include one or more plasmonic nanoparticles. In some embodiments, the plasmonic nanoparticles can include, without limitation, metal nanoparticles, magnetic nanoparticles, functionalized nanoparticles, functionalized magnetic nanoparticles, and combinations thereof.

In some embodiments, the detection particles include a plurality of plasmonic nanoparticles. In some embodiments, the plurality of plasmonic nanoparticles include plasmonic nanoparticles with different sizes. In some embodiments, the plasmonic nanoparticles are gold nanoparticles. In some embodiments, the plasmonic nanoparticles are arranged on the silica nanofibers in such a manner that the arranged plasmonic nanoparticles can enhance the electromagnetic field of the incident light at the vicinity of the surface of the plasmonic nanoparticles.

In some embodiments, the detection particles of the present disclosure may also include one or more analyte binding agents. In some embodiments, the analyte binding agent can include, without limitation, antibodies, aptamers, nucleic acids, small molecules, and combinations thereof.

The detection particles of the present disclosure may be associated with the silica nanofibers in various manners. For instance, in some embodiments, the detection particles of the present disclosure are non-randomly arranged on the silica nanofibers. In some embodiments, the detection particles are patterned on the silica nanofibers. In some embodiments, the detection particles are separated from one another at predetermined distances. In some embodiments, the predetermined distances have a spatial resolution as high as 6 nm. In some embodiments, the detection particles are within a hollow cavity of the silica nanofibers.

In some embodiments, the detection particles include a first particle and a second particle. In some embodiments, the first particle and the second particle exhibit different changes in a property upon interaction with one or more analytes, upon detection of one or more environmental conditions, or combinations thereof. In some embodiments, the first particle and the second particle are associated with the silica nanofibers in such a manner that the first particle is able to transfer energy to the second particle when the first particle is in an electronic excited state.

In some embodiments, the first particle is on an outer surface of the silica nanofibers, and the second particle is within a hollow cavity of the silica nanofibers. In some embodiments, the first particle includes fluorescein isothiocyanate (FITC) and the second particle includes Ru(2,2′-bipyridine)₃ (Ru(bpy)₃).

### Sensors

The compositions of the present disclosure may be incorporated as components of various devices. For instance, in some embodiments, the compositions of the present disclosure are a component of a sensor. As such, in some embodiments, the present disclosure pertains to sensors that include the compositions of the present disclosure.

In some embodiments, the sensors of the present disclosure include a surface that is associated with the silica nanofibers of the present disclosure, a light source positioned near the silica nanofibers and operational to transmit light to the one or more detection particles associated with the silica nanofibers, and a measuring device operational to measure light emitted from the detection particles associated with the silica nanofibers.

In some embodiments, the light source includes a laser. In some embodiments, the laser includes, without limitation, a gas laser, a solid-state laser, a fiber laser, a liquid laser, a semiconductor laser, and combinations thereof.

In some embodiments, the measuring device measures an emission wavelength peak from the light emitted from the detection particles associated with the silica nanofibers. In some embodiments, the measuring device includes a spectrometer.

In some embodiments, the sensors of the present disclosure also include an optical fiber. In some embodiments, the optical fiber is connected to the measuring device and positioned near the silica nanofibers.

The sensors of the present disclosure can include various types of surfaces. For instance, in some embodiments, the surfaces can include polymers. In some embodiments, the polymers are plasma treated. In some embodiments, the polymers include polystyrene. In some embodiments, the polystyrene is a plasma-treated polystyrene. In some embodiments, plasma treatment of a polymer surface modifies the surface, thereby resulting in a specifically targeted behavior and hydrophilicity, which allows the silica nanofibers to be bonded to the polymer (e.g., polystyrene) surface through hydrogen bonds.

In some embodiments illustrated in FIG. 1B, the sensors of the present disclosure are in the form of sensor 20. As set forth in FIG. 1B, sensor 20 generally includes: silica nanofibers 12 (e.g., silica nanofibers 12 shown in FIG. 1A); surface 18 associated with the silica nanofibers (e.g., surface 18 shown in FIG. 1A); light source 36 positioned near silica nanofibers 12 and operational to transmit light 30 to the one or more detection particles associated with the silica nanofibers (e.g., detection particles 14 and 16 shown in FIG. 1A); mirror 34 for reflecting the transmitted light 30 towards surface 18; lens 32 for focusing the transmitted light 30 onto silica nanofibers 12 on surface 18; measuring device 38 operational to measure lights 22 and 24 emitted from the detection particles associated with silica nanofibers 12; optical fiber 26 with tip 28, which is connected to measuring device 38 and positioned near silica nanofibers 12; and emission filter 40 for filtering the emitted light.

Sensing Analytes and/or Environmental Conditions

Additional embodiments of the present disclosure pertain to methods of sensing one or more analytes and/or one or more environmental conditions from a sample by utilizing the compositions and sensors of the present disclosure. In some embodiments illustrated in FIG. 1C, the methods of the present disclosure include: associating the sample with a composition of the present disclosure, which generally includes silica nanofibers and one or more detection particles associated with the silica nanofibers (step 40); detecting a change in a property of the detection particles (step 42); and correlating the change in the property of the detection particles to a presence or absence of one or more analytes, one or more environmental conditions, or combinations thereof (step 44). As set forth in more detail herein, the sensing methods of the present disclosure can have numerous variations.

### Samples

The methods of the present disclosure can sense various analytes and environmental conditions from various types of samples. For instance, in some embodiments, the sample can include, without limitation, a liquid, a gas, and combinations thereof. In some embodiments, the sample includes a liquid. In some embodiments, the sample includes an actual environment.

Associating a Sample with a Composition

Various methods may also be utilized to associate a sample with the compositions of the present disclosure. For instance, in some embodiments, the associating includes flowing the sample through the composition. In some embodiments, the associating includes incubating the sample with the composition. In some embodiments, the associating includes exposing the sample to the composition.

### Detecting a Change in a Property of the Detection Particles

Various changes in properties of the detection particles of the present disclosure can occur to identify the presence or absence of one or more analytes, one or more environmental conditions, or combinations thereof. For instance, in some embodiments, the detected change in the property of the one or more detection particles includes, without limitation, a change in optical intensity, a change in emission wavelength peak, a change in emission wavelength phase, fluorescence resonance energy transfer (FRET), a shift in localized surface plasmonic resonances (LSPR), and combinations thereof.

Various methods may be utilized to detect a change in a property of the one or more detection particles. For instance, in some embodiments, the detecting occurs by a method that includes, without limitation, visualization, microscopy, dark field microscopy, spectrometry, spectroscopy, colorimetric analysis, localized surface plasmon resonance (LSPR), nuclear magnetic resonance (NMR), surface plasmon resonance, electrochemistry, and combinations thereof. In some embodiments, the detecting includes visualizing a color or image change of the detection particles.

In some embodiments, the detecting includes: exposing the composition to a light source, where the exposing transmits light to the detection particles associated with the silica nanofibers, and utilizing a measuring device to measure light emitted from the detection particles associated with the silica nanofibers. In some embodiments, the measured light emitted includes a measured emission wavelength peak.

### Correlating

Various methods may also be utilized to correlate the change in the property of the detection particles to a presence or absence of one or more analytes, one or more environmental conditions, or combinations thereof. For instance, in some embodiments, the correlating includes comparing the change in the property of the particles with known properties in a database.

In some embodiments, the correlating includes correlating the change in the property of the detection particles to the presence of a concentration of the one or more analytes, the one or more environmental conditions, or combinations thereof. As such, in some embodiments, the methods of the present disclosure may be utilized to quantify the presence of one or more analytes, one or more environmental conditions, or combinations thereof.

In some embodiments, the correlating includes utilization of a machine-learning algorithm. In some embodiments, the correlating includes comparing the change in the property of the one or more detection particles with results from other existing detection particles.

In some embodiments, the detection particles of the silica nanofibers include a first particle and a second particle. In some embodiments, the first particle and the second particle exhibit different changes in a property upon interaction with the one or more analytes, upon detection of the one or more environmental conditions, or combinations thereof. In some embodiments, the changes in the property include changes in emission wavelength peaks of the first particle and the second particle. In some embodiments, the ratio of the change in the emission wavelength peak of the first particle relative to the change in the emission wavelength peak of the second particle is utilized to correlate the change in the property of the detection particles to the presence or absence of the one or more analytes, the one or more environmental conditions, or combinations thereof.

In some embodiments, the first particle and the second particle are associated with the silica nanofibers in such a manner that the first particle is able to transfer energy to the second particle when the first particle is in an electronic excited state. In some embodiments, the transfer of energy from the first particle to the second particle is utilized to correlate the change in the property of the detection particles to the presence or absence of the one or more analytes, the one or more environmental conditions, or combinations thereof.

In some embodiments, the first particle is on an outer surface of the silica nanofibers. In some embodiments, the second particle is within a hollow cavity of the silica nanofibers.

### Analytes and Environmental Conditions

The methods of the present disclosure may be utilized to sense various analytes and environmental conditions. In some embodiments, the methods of the present disclosure are utilized to sense one or more analytes from a sample. In some embodiments, the methods of the present disclosure are utilized to sense one or more environmental conditions from a sample. In some embodiments, the methods of the present disclosure are utilized to sense one or more analytes and one or more environmental conditions from a sample.

In some embodiments, the one or more analytes include, without limitation, environmental analytes, pollutants, biomolecules, cellular analytes, nucleic acids, DNA, single-stranded DNAs, double-stranded DNAs, RNAs, messenger RNAs (mRNA), proteins, antibodies, hormones, enzymes, antigens, cells, and combinations thereof. In some embodiments, the one or more analytes include a plurality of analytes.

In some embodiments, the one or more environmental conditions include, without limitation, cellular metabolism level, pH, temperature, cellular force, force, changes in biochemical environment near cells in the sample, and combinations thereof. In some embodiments, the one or more environmental conditions include pH of the environment. In some embodiments, the one or more environmental conditions include a plurality of environmental conditions.

### Monitoring of Samples

The methods of the present disclosure can detect or monitor a sample for various analytes and environmental conditions in various manners. For instance, in some embodiments, the detecting and/or correlating steps are conducted in real-time. In some embodiments, the detecting and/or correlating steps include continuous monitoring of a sample.

In some embodiments, the methods of the present disclosure can be repeated with various samples. In some embodiments, the methods of the present disclosure can be continually repeated. In some embodiments, the methods of the present disclosure can provide for real-time monitoring of the one or more analytes, one or more environmental conditions, or combinations thereof.

In some embodiments, the sensors of the present disclosure may be utilized to sense various analytes and environmental conditions in accordance with the method of the present disclosure. For instance, in some embodiments, sensor 20 shown in FIG. 1B may be utilized to sense various analytes and environmental conditions. In operation, light source 36 transmits light 30 to detection particles 14 and 16 associated with near silica nanofibers 12 on surface 18. During this process, mirror 34 reflects the transmitted light 30 towards surface 18 while lens 32 focuses the transmitted light 30 onto silica nanofibers 12 on surface 18.

As a result, detection particles 14 and 16 emit lights 22 and 24. The emitted lights are transmitted by tip 28 of optical fiber 26 to measuring device 38 after filtration through emission filter 40. Thereafter, measuring device 38 detects the emitted lights. The emitted lights may then be correlated to the presence or absence of one or more analytes, one or more environmental conditions, or combinations thereof.

### Fabrication of Compositions

Additional embodiments of the present disclosure pertain to methods of making the compositions of the present disclosure. In some embodiments illustrated in FIG. 1D, the methods of the present disclosure include: growing silica nanofibers from at least one precursor material (step 50); and associating one or more detection particles with the silica nanofibers (step 52). As set forth in more detail herein, the fabrication methods of the present disclosure can also have numerous variations.

### Silica Nanofiber Growth

The methods of the present disclosure may utilize various steps to grow silica nanofibers. For instance, in some embodiments, the growth occurs by hydrolysis of the at least one precursor material. In some embodiments, the at least one precursor material includes tetraethyl orthosilicate (TEOS).

Association of Detection Particles with Silica Nanofibers

Detection particles may be associated with silica nanofibers at various stage of growth. For instance, in some embodiments, the associating occurs during the growing of the silica nanofibers. In some embodiments, the associating occurs after the growing of the silica nanofibers. In some embodiments, the associating occurs during and after the growing of the silica nanofibers.

In some embodiments, the associating occurs by a method that includes, without limitation, coating, doping, covalent coupling, or combinations thereof. In some embodiments, the associating occurs by covalent coupling. In some embodiments, the covalent coupling includes the utilization of an alkyl alkoxysilane that couples the detection particles to the silica nanofibers.

The methods of the present disclosure can utilize various alkyl alkoxysilanes for coupling detection particles to silica nanofibers. For instance, in some embodiments, the alkyl alkoxysilane includes at least one amino group and at least one silanol group. In some embodiments, the amino group covalently bonds to the one or more detection particles through amide bonds while the silanol group couples to the silica nanofibers through a co-condensation reaction. In some embodiments, the alkyl alkoxysilane includes 3-aminopropyl)triethoxysilane (APTES).

Various detection particles may be associated with silica nanofibers. Suitable detection particles were described previously in this patent application. In some embodiments, the detection particles include a first particle and a second particle. In some embodiments, the first particle and the second particle exhibit different changes in a property upon interaction with one or more analytes, upon detection of one or more environmental conditions, or combinations thereof.

In some embodiments, the first particle becomes associated on an outer surface of the silica nanofibers. In some embodiments, the second particle becomes associated within a hollow cavity of the silica nanofibers. In some embodiments, the first particle becomes associated on an outer surface of the silica nanofibers after the growing step. In some embodiments, the second particle becomes associated within a hollow cavity of the silica nanofibers during the growing step. In some embodiments, the first particle includes fluorescein isothiocyanate (FITC) and the second particle includes Ru(2,2′-bipyridine)₃ (Ru(bpy)₃).

### Applications and Advantages

The present disclosure can have various advantages. For instance, in some embodiments, the compositions and sensors of the present disclosure can allow real-time and continuous monitoring of various analytes and environmental conditions. In some embodiments, the compositions and sensors of the present disclosure have multiplex sensing capability, thereby allowing them to detect different environmental conditions and analytes simultaneously.

As such, the compositions and sensors of the present disclosure can have numerous applications. For instance, in some embodiments, the compositions and sensors of the present disclosure can be utilized for real-time, continuous monitoring of various analytes and pH values of various environments.

### ADDITIONAL EMBODIMENTS

Reference will now be made to more specific embodiments of the present disclosure and experimental results that provide support for such embodiments. However, Applicants note that the disclosure below is for illustrative purposes only and is not intended to limit the scope of the claimed subject matter in any way.

### Example 1. Dual Fluorescence Hollow Silica Nanofibers for In Situ pH Monitoring Using an Optical Fiber

In this Example, applicant reports a sensitive and robust pH sensor based on dual fluorescence doped hollow silica nanofibers (hSNFs) for long-term pH monitoring. Fluorescein isothiocyanate (FITC) and Tris(2,2-bipyridyl)dichlororuthenium(II) hexahydrate (Ru(BPY)₃) were chosen as pH sensitive dye and reference dye, respectively. Two-step synthesized hSNFs from a reverse micelle system had an average length of 6.20 μm and an average diameter of 410 nm. The intensity ratio of FITC/Ru(BPY)₃ is used to calibrate with pH changes. Applicant has also developed an optical-fiber-based fluorescent detection system that enables feasible and highly efficient near-field fluorescent detection. The system has a fully automated fluorescent detection procedure, where components including the light source, detector, and data acquisition are all controlled by the computer. The results show that pH sensor works in a linear range of pH 4.6-pH 8.5 with a fast response time of less than 10 seconds. In addition, the as-prepared hSNFs-based pH sensors also have optimal long-term durability.

### Example 1.1. Materials

Fluorescein isothiocyanate (FITC), Tris(2,2′-bipyridyl)dichlororuthenium(II) hexahydrate (Ru(BPY)₃), 3-aminopropyl triethyloxylsilane (APTES), 1-Pentanol, tetraethyl orthosilicate (TEOS), trisodium citrate dihydrate, polyvinylpyrrolidone (PVP, 40 kDa), ammonium hydroxide (25 wt % aq.) and ethanol (200-proof) were purchased from Sigma-Aldrich. Water used was from a Milli-Q water ultrapure water purification system. All chemicals were used as received without any further purification.

A handheld high frequency generator (Electro-Technic Products, BD-20) was used for plasma treatment of the polymer surface. Transmission electron microscopy (TEM) was performed on a Tecnai F20ST field emission gun (FEG) transmission electron microscope operating at an accelerating voltage of 200 kV. Scanning electron microscopy (SEM) was performed on the Thermo Scientific Helios 5cx Dual Beam scanning electron microscope. The energy-dispersive X-ray spectroscopy (EDS) measurements were performed with the spectrometer attached on the Tecnai F20ST FEG electron microscope. Fluorescent images were taken by Olympus BX51 microscope.

### Example 1.2. Synthesis of hSNFs with Internally Doped Ru(BPY)₃

The Ru(BPY)₃ doped hSNFs were prepared following reported methods with modifications. In a typical reaction, 5 mL of 1-pentanal and PVP solution were first added in a 15 mL glass scintillation vial and mixed at 50° C. for 30 minutes. Then, 475 mL of ethanol, 100 mL of DI water and 40 mL of Ru(BPY)₃ solution (30 mM in water) were added to the mixed solution, and the mixture was vortexed for 1 min. Next 50 μL of 0.18 M aqueous trisodium citrate dihydrate was injected and vortexed for 1 min, followed by the addition of 100 μL of ammonia hydroxide (25 wt % aq.) with vortex mixing for 1 min. Lastly, 50 μL of TEOS was injected and then immediately vortexed for 20 s. The mixtures reacted without stirring at 70° C. overnight for 12 h. After the reaction, the solution was poured into a 15 mL centrifuge tube, and 1 mL of acetone was added to break the microemulsion. hSNFs were isolated by centrifuging (1500 rpm, 15 min), and washed with water and ethanol (1000 rpm, 5 min) for 3 times (ultrasonic cleaning, 5 min each time) to remove the surface impurities.

### Example 1.3. FITC Coating on the Surfaces of hSNFs

FITC was coated on the surfaces of hSNFs using a fluorescence coating method. 20 mg Ru(BPY)₃ doped hSNFs fabricated in the last step were dispersed in 2 mL ethanol in a 15 mL centrifuge tube. Next, 100 μL of ammonia hydroxide (25 wt % aq.), 100 μL of DI water and 20 μL of TEOS were added sequentially and vortexed for 20 s. 10 μL of FITC solution (15 mg FITC and 150 μL APTES reacted for 24 h in 1 mL of ethanol at room temperature) was then added. The above mixture was left without stirring at 40° C. for 8 h. The hSNFs doped with both FITC and Ru(BPY)₃ were isolated by centrifuging (700 rpm, 15 min), and washed 2 times with DI water (ultrasonic cleaning, 5 min each time) and 2 times with ethanol (ultrasonic cleaning, 5 min each time).

### Example 1.4. Deposition of the Dual Fluorophores Doped hSNFs

20 mg of the fabricated dual fluorescence hSNFs were dispersed in 2 mL ethanol. Petri dish (Ø60×H15 mm, polystyrene) was first plasma treated using the handheld high frequency generator for 1 min to induce a hydrophilic bottom surface by adding hydroxyl groups into hydrocarbon chains. Right after the plasma treatment, 50 μL of the dual fluorescence hSNFs solution were dispensed onto the center of petri dish, followed by spin-coating process (200 rpm for 5 s, and 400 rpm for 15 s).

### Example 1.5. pH Sensing Based on the Optical Fiber Readout System

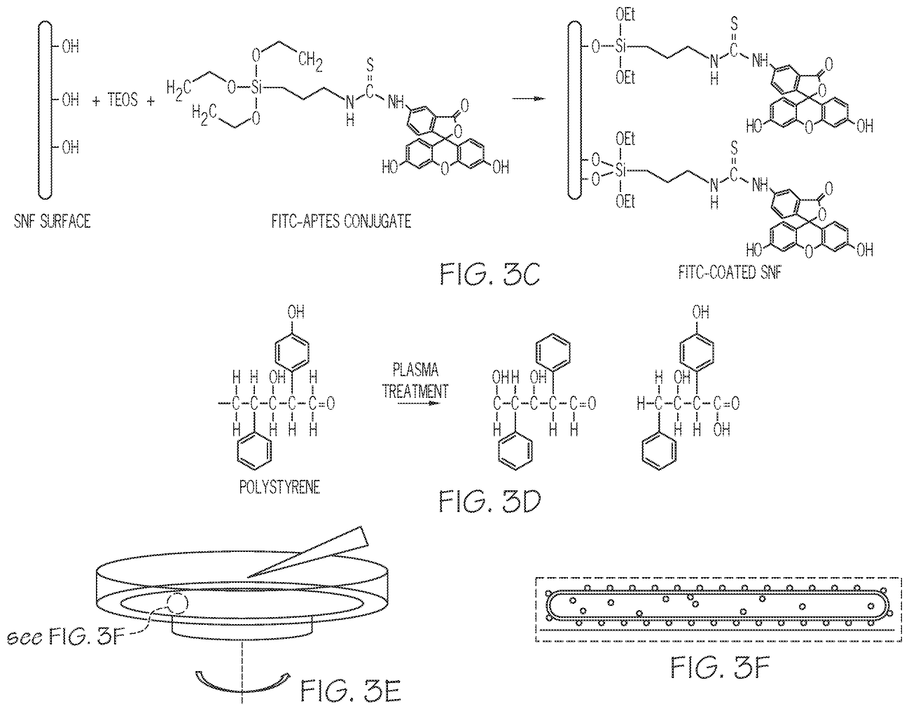

To easily read out the fluorescent spectrum of the hSNFs, Applicant has developed a customized fluorescent detection system based on optical fibers, as shown in FIG. 2. The system consists of a 488 nm excitation laser (Techhood), an inverted microscope with a 20× objective lens (Inverted Metallurgical Microscope, AmScope), a multimode optical fiber with a 105 μm diameter core (FG105UCA, Thorlabs), a 500 nm cut-on emission filter (FEL0500, Thorlabs), and an optical spectrometer (Flame Spectrometer, OceanOptics). The 488 nm laser was coupled into the inverted microscope and focused onto the sample by the 20× objective lens, which acted as the excitation light for the FITC and Ru(BPY)₃ fluorophores. The multimode fiber has a cleaved flat end and was pre-aligned to less than 3 μm above the silica nanofibers, as shown in the microscope image inset in FIG. 2. The optical fiber was tilted by a ˜45° angle with respect to the plane of the hSNFs coating to reduce the background light signal due to the transmitted excitation light. The emitted fluorescent light together with the scattered excitation light were collected by the multimode fiber and transmitted to an emission filter.

The emission filter blocked light lower than 500 nm, which includes the 488 nm excitation laser. This prevented the spectrometer to be overloaded by the strong scattered excitation light and the distortion of the fluorescent spectrum. Compared to fluorescent pH sensors based on microscope imaging, in which the fluorescent signal is read out in the form of pixel intensities, Applicant's fiber-based system can read out the whole spectrum of the fluorescent signal in real time. As will be detailed in this Example, real-time pH measurements are based on this acquired fluorescent spectrum.

### Example 1.6. Synthesis of the Dual Fluorescence hSNFs

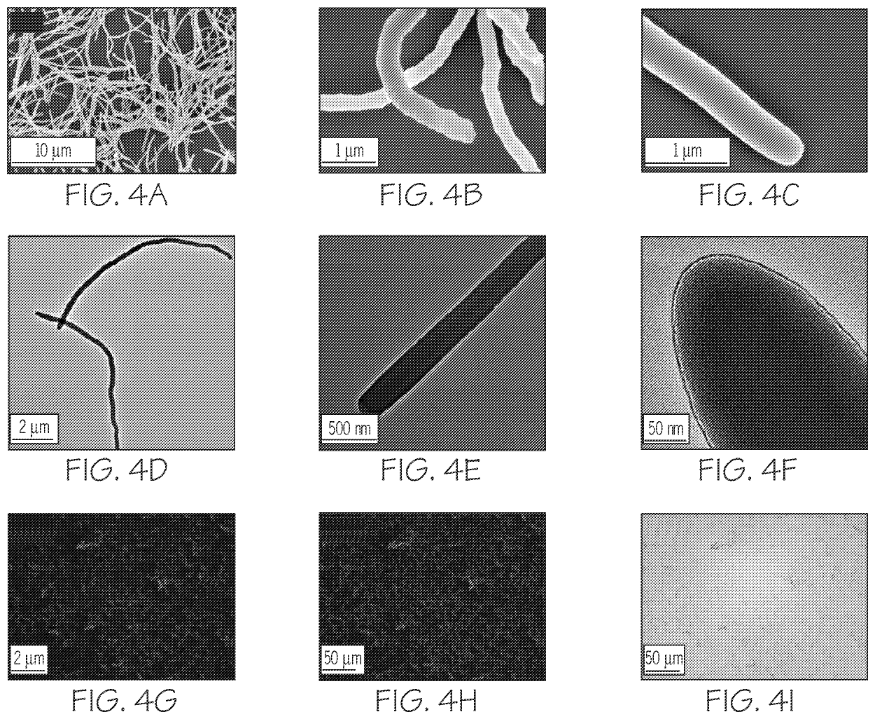

The synthesis of the dual fluorescence hSNFs is illustrated in FIGS. 3A-3F. The design is based on the consideration that the fluorophore Ru(BPY)₃ is doped into the hSNFs during TEOS hydrolysis acting as reference dye, while fluorophore FITC is coated on the surfaces of hSNFs enabling pH response. Due to the solution-liquid-solid (SLS) mechanism, microemulsion system is created by dispersing the catalyst liquid phase (water droplets) into solution phase (pentanol). Thereafter, the precursor (TEOS) diffuses from the solution phase into the liquid phase to introduce the produce of target monomers (silica oligomers).

In Applicant's experiments, hydrolyzed TEOS molecules (silica oligomers) formed on the droplet surface at high ammonia content. They were then deposited immediately as solid silica before reaching the droplet center, so that hSNFs were synthesized instead of solid silica nanorods.

The synthesis process can be summarized as follows: Firstly, Ru(BPY)₃ was dissolved in the water droplets in microemulsion and encapsulated inside the hollow center of hSNFs. Then, FITC-APTES conjugate was grafted onto silica matrix while additional TEOS was added to grow a thin silica film simultaneously. The hollow structure allows having most Ru(BPY)₃ inside SNFs, which prevented its interaction from the environment and made it a stable reference signal in the ratiometric measurement. Coating FITC outside allows it to respond to environmental pH variation.

### Example 1.7. Morphological and Optical Properties of the Silica Nanofibers

The average length of hSNFs before coating FITC was (6.20±1.59) μm and the average diameter was (238±51) nm. By adjusting reaction parameters (i.e., concentration of chemical reagents, temperature, and reaction time), Applicant achieved a relative long aspect ratio of 26.5.

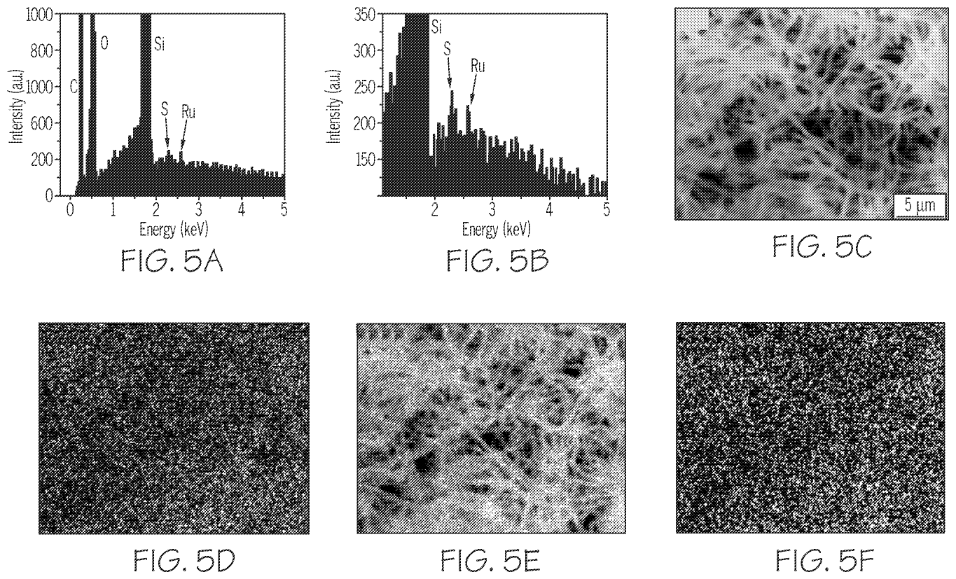

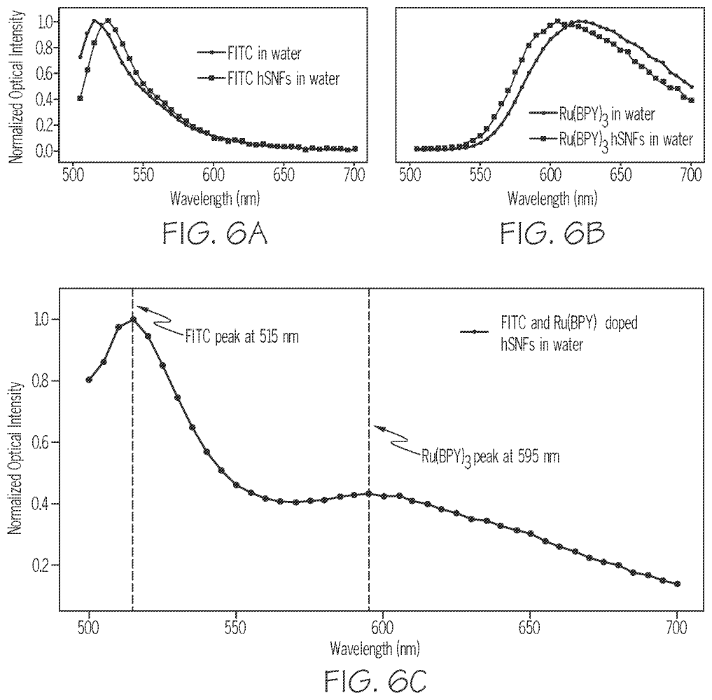

The shell thickness of hSNFs was about 41 nm (FIG. 4F). After coating with FITC, the average diameter of hSNFs was increased to around 410 nm. The energy-dispersive X-ray spectroscopy (EDS) was used to characterize the elements of the dual fluorescence hSNFs. As shown in FIGS. 5A-5F, the elements silicon (Si), carbon (C), oxygen (O), sulfur (S), and ruthenium (Ru) were confirmed via the EDS spectrum. The silicon elements were from the hSNFs. The sulfur element existed only in FITC molecules while the ruthenium element was only from Ru(BPY)₃ molecules.

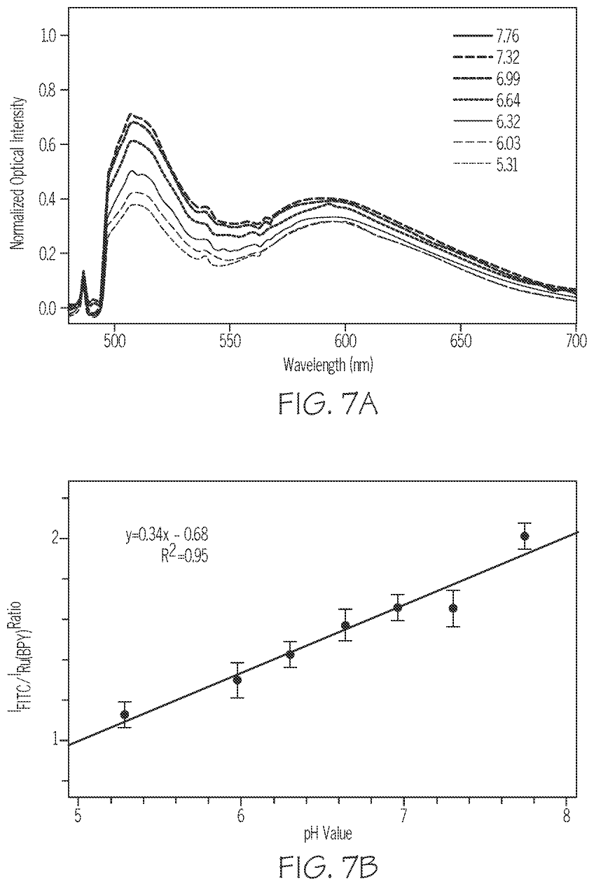

To further confirm that FITC and Ru(BPY)₃ fluorophores have been successfully doped to the hSNFs, and to identify the fluorescent peak wavelengths of the dual fluorescence hSNFs, Applicant conducted four emission spectrum analysis using the microplate reader (BioTek). FIGS. 6A and 6B compare the emission spectra of FITC and Ru(BPY)₃ fluorophores directly dissolved in water and that of hSNFs doped with only FITC or Ru(BPY)₃. These comparisons confirm that FITC and Ru(BPY)₃ fluorophores have been successfully doped to the hSNFs.

Applicant notes that the peak fluorescent wavelengths of both FITC and Ru(BPY)₃ have shifted after being coated to the SNFs. These shifts were due to the coupling of the transition dipole moment of the fluorophores with the silica nanofibers and the change of the fluorophores' molecular conformation. Emission spectrum of the dual fluorescence hSNFs was used to identify the fluorescence peaks of FITC and Ru(BPY)₃, as shown in FIG. 6C. The results show that the FITC and Ru(BPY)₃ peaks of the dual fluorescence hSNFs were at around 515 nm and 595 nm respectively. These peak wavelengths were used in the following pH sensing experiments to calculate the FITC and Ru(BPY)₃ intensity ratios.

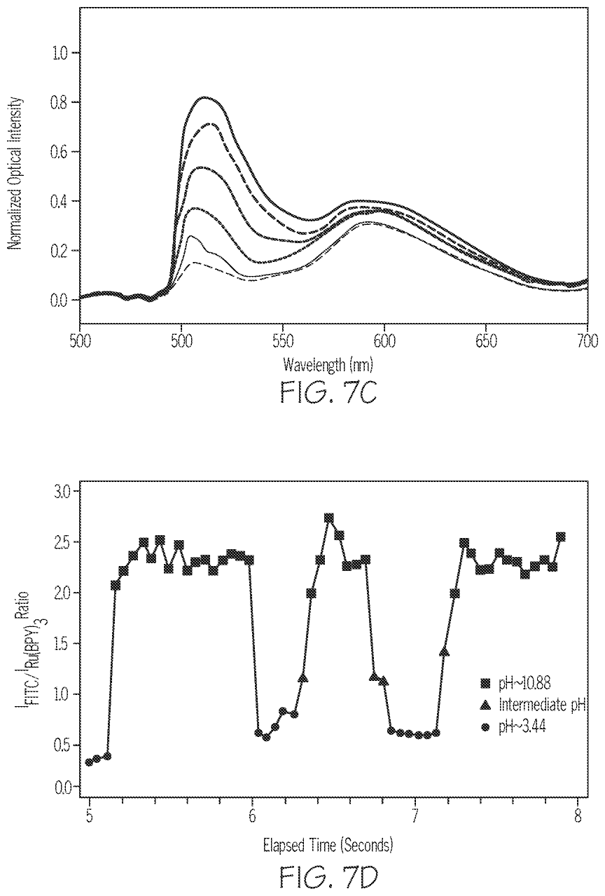

Applicant has experimentally characterized the pH responses of the dual fluorescence hSNFs using the optical characterization setup shown in FIG. 2. The characterization of the dual fluorescence hSNFs' pH sensitivity was shown in FIGS. 7A and 7B.

In the experiments, the environmental pH of the dual fluorescence hSNFs coated petri dish was changed from alkaline (pH=7.76) to acidy (pH=5.31) by adding 0.01M HCl solution to a diluted NaOH solution. Fluorescent spectra of the dual fluorescence hSNFs at seven different pH values were characterized. At each pH value, 10 spectra were recorded with an exposure time of 0.5 seconds. The averaged spectra at each pH value were plotted in FIG. 7A. The results show that, as the solution became more acidic, the intensity of the FITC induced fluorescent peak decreased, which matched Applicant's previous discussion of FITC's pH dependency. However, as shown in FIG. 7A, the pH independent Ru(BPY)₃ fluorophore had also shown a decrease in the fluorescent as the pH decreased. This decrease was due to photobleaching of the Ru(BPY)₃ by the 488 nm excitation laser.

Since such photobleaching also acted on the FITC, Applicant used a ratiometric characterization method to decouple the pH-induced and photobleaching-induced FITC fluorescent intensity decrease. In this method, the environmental pH change was characterized by the ratio of FITC over Ru(BPY)₃ intensity. FIG. 7B shows the calculated I_{FITC}/I_{Ru(BPY)3} ratio plotted against the pH values.

A linear regression model was used to characterize the relationship between the I_{FITC}/I_{Ru(BPY)3} ratio and the pH values. The fitting results show that the I_{FITC}/I_{Ru(BPY)3} ratio has a linear relationship with the pH value and a pH sensitivity of 0.34 [pH⁻¹]. The strength of the linear fitting was characterized using the R₂ value, which was calculated to be 0.95. It is noted that for pH in the range of 5.5 to 7.5, the I_{FITC}/I_{Ru(BPY)3} ratio had a much higher linearity of R₂>0.99 with respect to the pH values.

In many pH sensing applications, it is desirable that the sensor has good reversibility. Here, Applicant has experimentally characterized the dual fluorescence hSNFs' response to fluctuating pH values. In the experiment, the hSNFs were first deposited onto a petri dish using the method discussed above. Then 0.01M HCl was added into the petri dish to achieve the initial acidic environment of pH=3.44. The pH value of the solution was then abruptly changed between 3.44 and 10.88 in cycles by adding the concentrated HCl and NaOH solution intermittently, while the fluorescent light emitted by the hSNFs was recorded every 10 seconds with an exposure time of 0.5 seconds, as shown in FIG. 7C.

The spectra in FIG. 7C were separate into three categories, spectra measured when the solution was in acidic or alkaline equilibrium state, and the spectra measured when the pH of the solution was between 3.44 and 10.88. Here, the acidic and alkaline equilibrium states of the solution was determined using an electrical pH sensor, and by checking whether the pH of the solution fluctuates less than ±0.02 using a reference electrical sensor.

To differentiate the spectra measured at different pH states, and to differentiate the overlapping spectra at the same pH state, different colors were assigned to the spectra in FIG. 7C. Spectra taken when the solution was in acidic equilibrium state (pH-3.44) were assigned with different shades of blue to dark blue. The darker the blue color the later the spectra were taken. Similarly for spectra taken when the solution was in alkaline equilibrium state (pH-10.88), different shades of red to dark red were used to separate spectra taken at different times. A gray color was assigned to all the spectra taken when the solution had not reached equilibrium and was in an intermediate pH.

To better characterize the pH responsiveness and reversibility of the dual fluorescence hSNFs, the I_{FITC}/I_{Ru(BPY)3} ratio variations in FIG. 7C were calculated and were plotted in FIG. 7D. In the plot, the different color of the dots represents I_{FITC}/I_{Ru(BPY)3} ratios at different pH states. The blue dots correspond to measurements taken in the acidic equilibrium state (blue to dark blue spectra), the red dots correspond to measurements taken in the alkaline equilibrium state (red to dark red spectra), and the black dots correspond to the intermedia state where the pH was fluctuating (gray spectra).

This I_{FITC}/I_{Ru(BPY)3} ratio plot showed that during the acidic-alkaline cycles, the I_{FITC}/I_{Ru(BPY)3} ratio of the hSNFs maintained a highly reversible response even when the environmental pH was abruptly fluctuating. The fluctuation of the I_{FITC}/I_{Ru(BPY)3} ratio in FIG. 7D in the pH equilibrium state was mainly due to the local pH fluctuation near the optical fiber tip. The dual fluorescence hSNFs also showed a relatively short response time to the more than 7 pH value jumps at each cycle.

In all the cases, the response times were less than 30 seconds, and in some cases, the response times were less than 10 seconds. As mentioned above, the ratiometric characterization method based on the FITC and Ru(BPY)₃ fluorescent intensity ratio can effectively decouple the pH induced FITC fluorescent intensity variation from that caused by the photobleaching. Such decoupling is particularly beneficial in long-term measurements, where the accumulated exposure time is often long enough to induce photobleaching of the fluorophores.

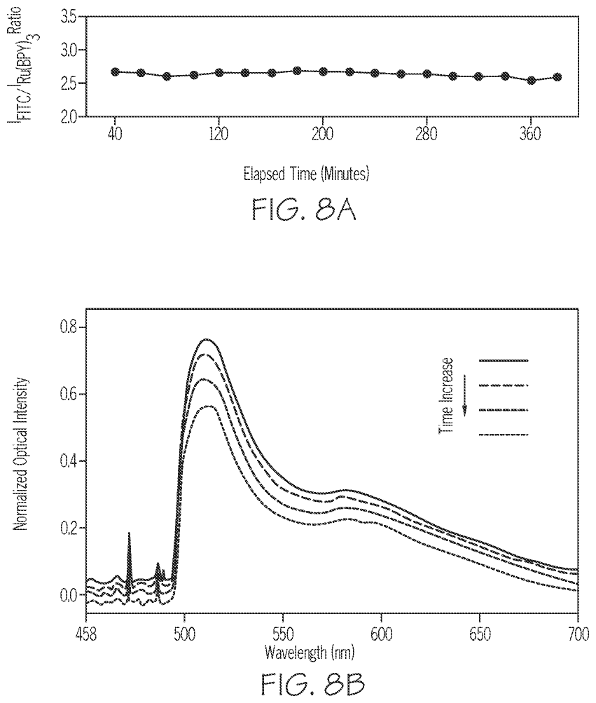

Here, Applicant experimentally demonstrated the effectiveness of the ratiometric characterization method in long-term pH sensing applications. In the experiment, dual fluorescence hSNFs coated petri dish was filled with pH=7 buffer. The fiber-based fluorescent detection system was used to read out the fluorescent spectrum every 20 minutes for over more than 6 hours. As shown by the recorded spectra in FIG. 8B, there were clear decreases in the fluorescent intensities for both FITC and Ru(BPY)₃, which were caused by the photobleaching.

However, as shown in FIG. 8A, the I_{FITC}/I_{Ru(BPY)3} ratio calculated based on the optical spectra had no significant decrease and had only minimum fluctuations, which matched the fact that the buffer solution had a constant pH. These results show that the I_{FITC}/I_{Ru(BPY)3} ratio was not affected by the photobleaching of the fluorophores, and faithfully represented the solution pH.

In summary, Applicant demonstrated in this Example a novel ratiometric fluorescence pH sensor based on hSNFs doped with FITC and Ru(BPY)₃ fluorophores. Using water-in-oil droplets created in microemulsion system, the dual fluorophore doped hSNFs with emission peaks at around 515 and 595 nm were synthesized in a straightforward fashion. The pH response of the dual fluorescence hSNFs was characterized using a customized optical fiber-based fluorescence detection system. The characterization results show that the dual fluorescence hSNFs have a pH sensitivity of 0.34 [pH⁻¹] and a response time of less than 10 seconds. Thanks to the ratiometric measurement approach based on the FITC and Ru(BPY)₃ fluorescent intensity ratio, the developed fluorescence pH sensor was able to achieve long term pH measurements with only minimum influence from photobleaching. Applicant have experimentally demonstrated that the sensor has a linear pH response between pH 4.6 to pH 8.5 and a long-term stability over more than 6 hours.

The photobleaching independent pH measurements of Applicant's hSNFs pH sensors made it ideal for applications that require long-term pH monitoring, such as the long-term cell metabolic profiling. Beyond the demonstrated pH sensing application, the developed rational synthesis method of hSNFs can potentially enable other sensing applications by rationally design the fluorophores doped to the hSNFs.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A composition comprising:
silica nanofibers; and
one or more detection particles associated with the silica nanofibers,
wherein at least one of the one or more detection particles are operational to exhibit a change in a property upon interaction with one or more analytes, upon detection of one or more environmental conditions, or combinations thereof, and
wherein the change in property is selected from the group consisting of a change in optical intensity, a change in emission wavelength peak, a change in emission wavelength phase, fluorescence resonance energy transfer (FRET), a shift in localized surface plasmonic resonances (LSPR), and combinations thereof.

2. The composition of claim 1, wherein the silica nanofibers are in the form of a matrix.

3. The composition of claim 1, wherein the silica nanofibers comprise a length of at least about 500 nm, a diameter of at least about 50 nm, a length to width aspect ratio of at least about 10, and a hollow cavity.

4. The composition of claim 1, wherein the detection particles are on an outer surface of the silica nanofibers.

5. The composition of claim 1, wherein the detection particles are within a hollow cavity of the silica nanofibers.

6. The composition of claim 1, wherein the detection particles are selected from the group consisting of chromophores, fluorophores, plasmonic nanoparticles, Ru(2,2′-bipyridine)₃ (Ru(bpy)₃), fluorescein isothiocyanate (FITC), quantum dots, and combinations thereof.

7. The composition of claim 1, wherein the detection particles comprise a first particle and a second particle, wherein the first particle and the second particle exhibit different changes in a property upon interaction with one or more analytes, upon detection of one or more environmental conditions, or combinations thereof.

8. The composition of claim 7, wherein the first particle and the second particle are associated with the silica nanofibers in such a manner that the first particle is able to transfer energy to the second particle when the first particle is in an electronic excited state.

9. The composition of claim 7, wherein the first particle is on an outer surface of the silica nanofibers, and wherein the second particle is within a hollow cavity of the silica nanofibers.

10. The composition of claim 9, wherein the first particle comprises fluorescein isothiocyanate (FITC) and the second particle comprises Ru(2,2′-bipyridine)₃ (Ru(bpy)₃).

11. The composition of claim 1, wherein the detection particles further comprise one or more analyte binding agents.

12. The composition of claim 1, wherein the composition is a component of a sensor, wherein the sensor comprises:
a surface associated with the silica nanofibers,
a light source positioned near the silica nanofibers and operational to transmit light to the detection particles associated with the silica nanofibers, wherein the light source comprises a laser,
a measuring device operational to measure light emitted from the detection particles associated with the silica nanofibers, wherein the measuring device comprises a spectrometer, and
an optical fiber, wherein the optical fiber is connected to the measuring device and positioned near the silica nanofibers.

13-15. (canceled)

16. A method of sensing one or more analytes, one or more environmental conditions, or combinations thereof from a sample, said method comprising:
associating the sample with a composition, wherein the composition comprises:
silica nanofibers, and
one or more detection particles associated with the silica nanofibers;
detecting a change in a property of the detection particles; and
correlating the change in the property of the detection particles to a presence or absence of one or more analytes, one or more environmental conditions, or combinations thereof.

17. The method of claim 16, wherein the change in the property is selected from the group consisting of a change in optical intensity, a change in emission wavelength peak, a change in emission wavelength phase, fluorescence resonance energy transfer (FRET), a shift in localized surface plasmonic resonances (LSPR), and combinations thereof.

18. The method of claim 16, wherein the detection particles are selected from the group consisting of chromophores, fluorophores, plasmonic nanoparticles, Ru(2,2′-bipyridine)₃ (Ru(bpy)₃), fluorescein isothiocyanate (FITC), quantum dots, and combinations thereof.

19. The method of claim 16, wherein the detection particles further comprise one or more analyte binding agents.

20. The method of claim 16, wherein the detection particles comprise a first particle and a second particle.

21. The method of claim 20, wherein the first particle and the second particle exhibit different changes in a property upon interaction with the one or more analytes, upon detection of the one or more environmental conditions, or combinations thereof.

22. The method of claim 20, wherein the changes in the property comprise changes in emission wavelength peaks of the first particle and the second particle, and wherein the ratio of the change in the emission wavelength peak of the first particle relative to the change in the emission wavelength peak of the second particle is utilized to correlate the change in the property of the detection particles to the presence or absence of the one or more analytes, the one or more environmental conditions, or combinations thereof.

23. The method of claim 20, wherein the first particle and the second particle are associated with the silica nanofibers in such a manner that the first particle is able to transfer energy to the second particle when the first particle is in an electronic excited state, and wherein the transfer of energy from the first particle to the second particle is utilized to correlate the change in the property of the detection particles to the presence or absence of the one or more analytes, the one or more environmental conditions, or combinations thereof.

24. The method of claim 20, wherein the first particle is on an outer surface of the silica nanofibers, and wherein the second particle is within a hollow cavity of the silica nanofibers.

25. The method of claim 24, wherein the first particle comprises fluorescein isothiocyanate (FITC) and the second particle comprises Ru(2,2′-bipyridine)₃ (Ru(bpy)₃).

26. The method of claim 16, wherein the detecting occurs by a method selected from the group consisting of visualization, microscopy, dark field microscopy, spectrometry, spectroscopy, colorimetric analysis, localized surface plasmon resonance (LSPR), nuclear magnetic resonance (NMR), surface plasmon resonance, electrochemistry, visualizing a color or image change of the detection particles, and combinations thereof.

27. (canceled)

28. The method of claim 16, wherein the detecting comprises:
exposing the composition to a light source, wherein the exposing transmits light to the detection particles associated with the silica nanofibers, and
utilizing a measuring device to measure light emitted from the detection particles associated with the silica nanofibers, wherein the measured light emitted comprises a measured emission wavelength peak.

29. (canceled)

30. The method of claim 16, wherein the correlating comprises; comparing the change in the property of the particles with known properties in a database; correlating the change in the property of the detection particles to the presence of a concentration of the one or more analytes, the one or more environmental conditions, or combinations thereof; or combinations thereof.

31. (canceled)

32. The method of claim 16, wherein the method is utilized to sense one or more analytes from the sample, wherein the one or more analytes is selected from the group consisting of environmental analytes, pollutants, biomolecules, cellular analytes, nucleic acids, DNA, single-stranded DNAs, double-stranded DNAs, RNAs, messenger RNAs (mRNA), proteins, antibodies, hormones, enzymes, antigens, cells, and combinations thereof.

33. (canceled)

34. The method of claim 16, wherein the method is utilized to sense one or more environmental conditions from the sample, wherein the one or more environmental conditions is selected from the group consisting of cellular metabolism level, pH, temperature, cellular force, force, changes in biochemical environment near cells in the sample, and combinations thereof.

35. (canceled)

36. The method of claim 34, wherein the one or more environmental conditions comprise pH of the environment.

37-48. (canceled)
